(19) United States
(12) Patent Application Publication
(10) Pub. No.: US 2025/0000987 A1
(43) Pub. Date: Jan. 2, 2025

(54) RETINOIC ACID COMPOUND, PHARMACEUTICAL COMPOSITION THEREOF AND USE THEREOF

(71) Applicant: Mastery Biotech CO., LTD., Taipei City (TW)

(72) Inventors: Ching-Yu Chen, Taichung City (TW); Bo-Lin Lin, Taipei City (TW)

(21) Appl. No.: 18/825,870

(22) Filed: Sep. 5, 2024

Related U.S. Application Data
(63) Continuation of application No. PCT/CN2022/079791, filed on Mar. 8, 2022.

(51) Int. Cl. A61K 47/54; A61K 9/127; A61K 31/203; A61K 33/06; A61K 33/30; A61P 31/14

(52) U.S. Cl. CPC A61K 47/549; A61K 9/127; A61K 31/203; A61K 33/06; A61K 33/30; A61P 31/14

(57) ABSTRACT

A compound or a pharmaceutically acceptable salt thereof, and a pharmaceutical composition thereof are provided, wherein the compound includes a retinoic acid conjugated with a carbohydrate. In addition, use of the compound or the pharmaceutically acceptable salt thereof or the pharmaceutical composition in the manufacture of a medicament for inhibiting infection or replication of a virus or for treating a cancer is also provided.

### CROSS-REFERENCE TO RELATED APPLICATION(S)

This application is a continuation of PCT/CN2022/079791, filed Mar. 8, 2022. The contents of the above-identified application are incorporated herein by reference in their entirety.

### FIELD OF INVENTION

The present disclosure relates to a novel retinoic acid compound, a pharmaceutical composition including thereof, and use thereof in the manufacture of a medicament for inhibiting virus infection or replication or for treating a cancer.

### BACKGROUND OF THE INVENTION

Coronaviruses are a group of positive-sense, single strand RNA viruses belonging to the Coronaviridae family, which includes seven species/strains that infect humans, i.e., severe acute respiratory syndrome coronavirus (SARS-COV), severe acute respiratory syndrome coronavirus 2 (SARS-COV-2), Middle East respiratory syndrome coronavirus (MERS-COV), human coronavirus 229E (HcoV-229E), human coronavirus OC43 (HCoV-OC43), human coronavirus NL63 (HCoV-NL63), human coronavirus HKU (HCoV-HKU1). Notably, SARS-COV-2 is identified as the viral strain causing the pandemic of coronavirus disease 2019 (COVID-19).

The SARS-COV-2 genome shares high sequence identity with that of SARS-COV. Both of SARS-COV-2 and SARS-COV critically rely on the activity of two viral proteases, namely, the 3C-like protease (3CLpro, also known as main protease (Mpro) or non-structural protein 5 (nsp5)) and the papain-like protease (PLpro, which is the protease domain of non-structural protein 3 (nsp3)), to achieve virus proliferation cycle and viral spread. It has been reported that all-trans-retinoic acid (ATRA, also called Vitamin A acid or Tretinoin) may be considered as a potential therapeutic agent against SARS-COV-2 by inhibiting 3CLpro activity.

Apart from 3CLpro, PLpro is also a potential target since such enzyme plays an essential role in cleavage and maturation of viral polyproteins, assembly of the replicase-transcriptase complex, and disruption of host responses. Even though the primary function of Mpro and PLpro is to process the viral polyprotein in a coordinated manner, PLpro has an additional function of stripping ubiquitin and IFN-stimulatory gene factor 15 (ISG15) from host-cell proteins to enable coronaviruses to avoid host innate immune responses. That is, PLpro not only relates to viral replication, but also is associated with dysregulation of signaling cascades in infected cells which gives rise to cell death in surrounding, uninfected cells. Therefore, drugs are designed to inhibit the function of PLpro also has the potential to fight against SARS-COV-2. Recently, in addition to ATRA, some researchers have reported its derivative, 13-cis-retinoic acid (also called isotretinoin, being a potential PLpro inhibitor) is a potential PLpro inhibitor and may be used for treatment of COVID-19 caused by SARS-COV-2.

Furthermore, retinoic acids including ATRA and 13-cis-retinoic acid are promising compounds for treatment of a variety of cancers because of its specific effects on cell proliferation, differentiation, and apoptosis, as well as its low toxicity. Retinoic acid receptors in human cell nuclei have been discovered by biochemists and found not mutated in cancer cells, and thus, retinoic acids could potentially exert its anticancer effects in many malignancies. For example, it was found that in children with high-risk neuroblastoma, treatment with 13-cis-retinoic acid can reduce the risk of the cancer coming back after high-dose chemotherapy and stem cell transplant. ATRA has been studied in combination with other drugs in various cancers and precancerous lesions. A number of clinical trials using ATRA as a part of combination therapy are currently underway. For instance, ATRA with different interferons (IFN) have been shown to enhance the effects of both drugs and lead to growth inhibition and cell death in tumor cell lines. Nevertheless, to unleash the therapeutic potentials of retinoic acids, many studies emphasize the need for better understanding of the mechanisms that block retinoic acid signaling and retinoic acid regulated gene expression in cancer, such as acute myeloid leukemia (AML). It is evident that combinatorial therapies targeting multiple gene silencing mechanisms may be the most effective strategy in reactivating ATRA-sensitive gene expression and differentiation of AML cells, as well as mediating anticancer activities of ATRA in general. Currently, the identification of classes of proteins that control gene expression via histone and DNA modifications is driving the development of new therapeutic agents, so-called epigenetic drugs that alter chromatin structure. However, these epigenetic modifying drugs have been shown to be only partly effective against different cancers when used alone.

### SUMMARY OF THE INVENTION

Based on the reasons above, the present invention provides a novel retinoic acid compound, and a pharmaceutical composition including thereof, which are capable of effectively inhibiting virus infection or replication, or treating a cancer.

In an aspect of the present invention, a compound or a pharmaceutically acceptable salt thereof is provided, wherein the compound includes a retinoic acid conjugated with a carbohydrate.

Preferably, the compound is represented by formula (I):

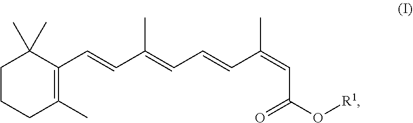

wherein R¹ is a substituted or unsubstituted functional group of the carbohydrate.

Preferably, the carbohydrate is selected from a group consisting of a monosaccharide, a disaccharide, an oligosaccharide, and a polysaccharide.

Preferably, the oligosaccharide is monooligosaccharide or heterooligosaccharide; and the polysaccharide is monopolysaccharide or heteropolysaccharide.

Preferably, the carbohydrate includes glucose, fructose, galactose, mannose, sucrose, lactose, maltose, β-1,3/1,6-glucan oligosaccharides, raffinose, stachyose, verbascose, fructooligosaccharides, starch, glycogen, cellulose, or any combination thereof.

Preferably, the compound or the pharmaceutically acceptable salt thereof is encapsulated by a liposome.

In another aspect of the present invention, a pharmaceutical composition is provided, including the compound or the pharmaceutically acceptable salt thereof described above and a pharmaceutically acceptable carrier.

Preferably, the pharmaceutically acceptable carrier includes a liposome, and the compound or the pharmaceutically acceptable salt thereof is encapsulated by the liposome.

Preferably, the pharmaceutical composition further includes a metal ion.

Preferably, the metal ion includes a monovalent ion, a divalent ion, or a combination thereof. More preferably, the monovalent ion includes K⁺, Na⁺, or a combination thereof; and the divalent ion includes Zn²⁺, Mg²⁺, Cu²⁺, Mn²⁺, Ca²⁺, Fe²⁺, or any combination thereof.

Preferably, the pharmaceutically acceptable carrier includes a liposome, and the metal ion is encapsulated by the liposome.

Preferably, the compound or the pharmaceutically acceptable salt thereof and the metal ion are individually or simultaneously encapsulated by the liposome.

In yet another aspect, the present invention provides use of said compound or said pharmaceutically acceptable salt thereof or said pharmaceutical composition in the manufacture of a medicament for inhibiting infection or replication of a virus.

In further another aspect, the present invention provides use of said compound or said pharmaceutically acceptable salt thereof or said pharmaceutical composition in the manufacture of a medicament for treating a cancer.

Therefore, the present invention at least provides the following advantages:
1. The claimed compound, which is a retinoic acid conjugated with a carbohydrate, and the pharmaceutical composition thereof can enhance the inhibitory ability of virus infection and/or or replication in comparison with a retinoic acid only.
2. The claimed compound and the pharmaceutical composition thereof can be served as a potential drug against a coronavirus, in particular, SARS-COV-2 that causes COVID-19.
3. The claimed compound and the pharmaceutical composition thereof also can enhance the ability to inhibit cancer cells and thus effectively treat various cancers, such as lung cancer, ovarian cancer, breast cancer, pancreatic cancer, colon cancer, and liver cancer.

### BRIEF DESCRIPTION OF THE DRAWINGS

FIG. 1 illustrates a liquid chromatography-mass spectrometry (LC-MS) analysis result of the compound (Ia) according to an embodiment of the present invention.

FIG. 2 illustrates a curve diagram of the absorbance versus time in SARS-COV-2 PLPro inhibition assay of the novel retinoic acid compound according to an embodiment of the present invention.

FIG. 3 illustrates a diagram of TCID₅₀ showing the antiviral effect of the novel retinoic acid compound in SARS-COV-2 animal model according to an embodiment of the present invention.

FIG. 4 illustrates a curve diagram of cell viability showing the cytotoxicity effect of the novel retinoic acid compound on the AsPC-1 cancer cell line according to an embodiment of the present invention.

FIG. 5 illustrates a curve diagram of cell viability showing the cytotoxicity effect of the novel retinoic acid compound on the MDA-MB-231 cancer cell line according to an embodiment of the present invention.

FIG. 6 illustrates a curve diagram of cell viability showing the cytotoxicity effect of the novel retinoic acid compound on the HCT-116 cancer cell line according to an embodiment of the present invention.

FIG. 7 illustrates a curve diagram of cell viability showing the cytotoxicity effect of the novel retinoic acid compound on the Huh-7 cancer cell line according to an embodiment of the present invention.

FIG. 8 illustrates a curve diagram of cell viability showing the cytotoxicity effect of the novel retinoic acid compound on the SKOV-3 cancer cell line according to an embodiment of the present invention.

FIG. 9 illustrates a curve diagram of cell viability showing the cytotoxicity effect of the novel retinoic acid compound on the A549 cancer cell line according to an embodiment of the present invention.

FIG. 10 illustrates a curve diagram of cell viability showing the cytotoxicity effect of the novel retinoic acid compound on the H460 cancer cell line according to an embodiment of the present invention.

FIG. 11 illustrates a curve diagram of the absorbance versus time in SARS-COV-2 PLPro inhibition assay of the second pharmaceutical compositions according to an embodiment of the present invention.

FIG. 12 illustrates a diagram of TCID₅₀ showing the antiviral effect of the second pharmaceutical compositions in SARS-COV-2 animal model according to an embodiment of the present invention.

### DETAILED DESCRIPTION

While the present invention is susceptible to various modifications and alternative forms, specific embodiments are shown by way of example in the drawings and described in detail. It should be understood, however, that the description is not intended to limit the present invention to the specific embodiments, but, on the contrary, the present invention is to cover all modifications, equivalents, and alternatives that fall within the spirit and scope of the present invention.

### Definitions

Unless otherwise defined, all terms (including technical and scientific terms) used herein have the same meaning as commonly understood by those of ordinary skill in the art to which the present invention pertains. It will be further understood that terms defined in commonly used dictionaries should be interpreted as having a meaning that is consistent with their meaning in the context of the related art and will not be interpreted in an idealized or overly formal sense unless expressly so defined herein.

The terminology used herein is for the purpose of describing particular embodiments only and is not intended to be limiting of embodiments of the present invention. As used herein, the singular forms “a,” “an,” and “the” are intended to include the plural forms as well, unless the context clearly indicates otherwise. It will be further understood that the terms “comprises,” “comprising,” “includes,” and/or “including,” when used herein, specify the presence of stated features, integers, steps, operations, elements, parts, and/or combinations thereof, but do not preclude the presence or addition of one or more other features, integers, steps, operations, elements, parts, and/or combinations thereof.

The term “subject” herein refers to a mammal, for whom diagnosis, prognosis, or therapy is desired. Generally, the mammal is a human. In certain embodiments, the mammal may refer to a non-human mammal used in for example, screening, characterizing, and evaluating drugs and therapies, such as non-human primates, cows, horses, goats, sheep, dogs, cats, rabbits, pigs, mice or rats.

The term “administration” or “administered” herein refers to introducing, providing or delivering a pre-determined active ingredient to a subject by any suitable routes to perform its intended function.

The term “cancer” herein refers to leukemias, lymphomas, carcinomas, sarcomas, and other malignant tumors of potentially unlimited growth that can expand locally by invasion and systemically by metastasis. Examples of cancers include, but are not limited to, cancer of ovary, adrenal gland, bone, brain, breast, bronchi, colon and/or rectum, gallbladder, head and neck, kidneys, larynx, liver, lung, neural tissue, pancreas, prostate, parathyroid, skin, stomach, and thyroid. Certain other examples of cancers include cholangiocarcinoma, acute and chronic lymphocytic and granulocytic tumors, adenocarcinoma, adenoma, basal cell carcinoma, cervical dysplasia and in situ carcinoma, Ewing's sarcoma, epidermoid carcinomas, giant cell tumor, glioblastoma multiforma, hairy-cell tumor, intestinal ganglioncuroma, hyperplastic corneal nerve tumor, islet cell carcinoma, Kaposi's sarcoma, leiomyoma, malignant carcinoid, malignant melanomas, malignant hypercalcemia, marfanoid habitus tumor, medullary carcinoma, metastatic skin carcinoma, mucosal neuroma, myeloma, mycosis fungoides, neuroblastoma, osteosarcoma, pheochromocytoma, polycythermia vera, primary brain tumor, small-cell lung tumor, squamous cell carcinoma of both ulcerating and papillary type, hyperplasia, seminoma, soft tissue sarcoma, retinoblastoma, rhabdomyosarcoma, renal cell tumor, topical skin lesion, veticulum cell sarcoma, and Wilm's tumor.

The term “oligosaccharide” herein refers to a carbohydrate comprised of a few number of monosaccharides, usually about three to ten monosaccharide units. Wherein, an oligosaccharide with one type of monosaccharide subunit is called monooligosaccharide; an oligosaccharide with more than one type of monosaccharide subunit is called heterooligosaccharide.

The term “polysaccharide” herein refers to a carbohydrate comprised of a large number of monosaccharide units. Wherein, a polysaccharide with one type of monosaccharide subunit is called monopolysaccharide; a polysaccharide with more than one type of monosaccharide subunit is called heteropolysaccharide.

The term “liposome” herein refers to a particle characterized by having an aqueous interior space sequestered from an outer medium by a membrane of one or more bilayers forming a vesicle. The major types of liposome are multilamellar vesicles (MLVs, with several lamellar phase lipid bilayers), small unilamellar liposome vesicles (SUVs, with single lipid bilayer) and large unilamellar vesicle (LUVs, with single lipid bilayer). Bilayer membranes of uni- or multi-lamellar vesicles are typically formed by lipids, i.e., amphiphilic molecules of synthetic or natural origin that include spatially separated hydrophobic and hydrophilic domains.

### DESCRIPTIVE EMBODIMENTS

Hereinafter, exemplary embodiments of the present invention will be described in greater detail with reference to the accompanying drawings.

In an embodiment, a compound or a pharmaceutically acceptable salt thereof, or a pharmaceutical composition including the compound (called “first” pharmaceutical composition below) is provided, wherein the compound includes a retinoic acid conjugated with a carbohydrate.

In an exemplary embodiment, the compound is represented by formula (I):

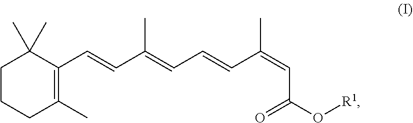

wherein R¹ is a substituted or unsubstituted functional group of the carbohydrate.

In certain embodiments, the concentration of the compound including the retinoic acid conjugated with the carbohydrate can be, but not limited to, from 0.1 μM to 10 mM, from 0.1 μM to 1 mM, from 0.1 μM to 500 μM, from 0.1 μM to 250 μM, from 0.1 μM to 100 μM, from 0.1 μM to 50 μM, from 1 μM to 10 mM, from 1 μM to 1 mM, from 1 μM to 500 μM, from 1 μM to 250 μM, from 1 μM to 100 μM, from 1 μM to 50 μM, from 10 μM to 10 mM, from 10 μM to 1 mM, from 10 μM to 500 μM, from 10 μM to 250 μM, from 10 μM to 100 μM, or from 10 μM to 50 μM.

In another embodiment, a pharmaceutical composition including a retinoic acid and a carbohydrate (called “second” pharmaceutical composition below) is provided. In certain embodiments, the retinoic acid is 13-cis-retinoic acid (also called isotretinoin).

In certain embodiments, the concentration of the retinoic acid can be, but not limited to, from 1 μM to 10 mM, from 1 μM to 1 mM, from 1 μM to 500 μM, from 1 μM to 250μ, from 1 μM to 100μ, from 1 μM to 50 μM, from 10 μM to 10 mM, from 10 μM to 1 mM, from 10 μM to 500 μM, from 10 μM to 250 μM, from 10 μM to 100 μM, or from 10 μM to 50 μM.

In certain embodiments, the carbohydrate may be a monosaccharide, a disaccharide, an oligosaccharide, or a polysaccharide. The oligosaccharide may be monooligosaccharide or heterooligosaccharide. The polysaccharide may be monopolysaccharide or heteropolysaccharide. The monosaccharide may be selected from, but not limited to, glucose, fructose, galactose and mannose. The disaccharide may be selected from, but not limited to, sucrose, lactose and maltose. The oligosaccharide may be selected from, but not limited to, β-1,3/1,6-glucan oligosaccharides, raffinose, stachyose, verbascose, fructooligosaccharides. The polysaccharide may be selected from, but not limited to, starch, glycogen and cellulose.

In certain embodiments, the concentration of the carbohydrate can be, but not limited to, from 0.1 μM to 200 mM, from 0.1 μM to 150 mM, from 0.1 μM to 100 mM, from 0.1 μM to 10 mM, from 0.1 μM to 1 mM, from 0.1 μM to 500 μM, from 0.1 μM to 250 μM, from 0.1 μM to 100 μM, from 0.1 μM to 50 μM, from 1 μM to 200 mM, from 1 μM to 150 mM, from 1 μM to 100 mM, from 1 μM to 10 mM, from 1 μM to 1 mM, from 1 μM to 500 μM, from 1 μM to 250 μM, from 1 μM to 100 μM, from 1 μM to 50 μM, from 10 μM to 200 mM, from 10 μM to 150 mM, from 10 μM to 100 mM, from 10 μM to 10 mM, from 10 μM to 1 mM, from 10 μM to 500 μM, from 10 μM to 250μ, from 10 μM to 100μ, or from 10 μM to 50 μM.

In certain embodiments, the molar ratio of the retinoic acid to the carbohydrate in the second pharmaceutical composition may be about 1:10⁻³-100, 1:10⁻³-20, 1:1-20, or 1:1-10.

In certain embodiments, the first and second pharmaceutical compositions each optionally further include a metal ion. Preferably, the metal ion includes a monovalent ion, a divalent ion, or a combination thereof. In certain embodiments, the monovalent ion includes K⁺, Na⁺, or a combination thereof; and the divalent ion includes Zn²⁺, Mg²⁺, Cu²⁺, Mn²⁺, Ca²⁺, Fe²⁺, or any combination thereof.

In certain embodiments, the concentration of the metal ion can be, from 1 μM to 300 mM, from 1 μM to 250 mM, from 1 μM to 200 mM, from 1 μM to 150 mM, from 1 μM to 100 mM, from 1 μM to 10 mM, from 1 μM to 1 mM, from 1 μM to 500 μM, from 1 μM to 250 μM, from 1 μM to 100 μM, from 1 μM to 50 μM, from 10 μM to 300 mM, from 10 μM to 250 mM, from 10 μM to 200 mM, from 10 μM to 150 mM, from 10 μM to 100 mM, from 10 μM to 10 mM, from 10 μM to 1 mM, from 10 μM to 500 μM, from 10 μM to 250μ, from 10 μM to 100 μM, or from 10 μM to 50 μM.

In certain embodiments, the molar ratio of the compound and the metal ion in the first pharmaceutical composition may be about 1:10⁻³-10³, 1:0.1-20, 1:0.1-10, or 1:1-10.

In certain embodiments, the molar ratio of the retinoic acid, the carbohydrate and the metal ion in the second pharmaceutical composition may be about 1:10⁻⁴-20:10⁻⁴-10³, 1:10⁻⁴-20:10⁻³-10³, 1:10⁻⁴-20:10⁻³-20, 1:10⁻⁴-20:0.1-20, 1:10⁻⁴-20:1-10, 1:0.1-20:10⁻⁴-10³, 1:0.1-20:10⁻³-10³, 1:0.1-20:10⁻³-20, 1:0.1-20:0.1-20, 1:0.1-20:1-20, or 1:0.1-20:1-10, 1:0.1-1:10⁻⁴-10³, 1:0.1-1:10⁻³-10³, 1:0.1-1:10⁻³-20, 1:0.1-1:0.1-20, 1:0.1-1:1-20, or 1:0.1-1:1-10.

In certain embodiments, the first and second pharmaceutical compositions each optionally further include a pharmaceutically acceptable carrier. The pharmaceutically acceptable carrier that is widely employed in the art of drug-manufacturing. Examples of the pharmaceutically acceptable carrier may include, but are not limited to, liposomes, excipients, adjuvants, solvents, buffers, emulsifiers, suspending agents, decomposers, disintegrating agents, dispersing agents, binding agents, stabilizing agents, chelating agents, diluents, gelling agents, preservatives, wetting agents, lubricants, absorption delaying agents, and the like. The choice and amount of the pharmaceutically acceptable carrier are within the expertise of those skilled in the art.

In certain embodiments, the pharmaceutically acceptable carrier is a liposome. The compound or the pharmaceutically acceptable salt thereof is encapsulated by the liposome. The compound or the pharmaceutically acceptable salt thereof and the metal ion in the first pharmaceutical composition may be individually or simultaneously encapsulated by the liposome. The retinoic acid and the carbohydrate in the second pharmaceutical composition may be individually or simultaneously encapsulated by the liposome. All or at the least two of the retinoic acid, the carbohydrate and the metal ion in the second pharmaceutical composition may be individually or simultaneously encapsulated by the liposome.

Exemplary liposomes may be neutrally, positively or negatively charged liposomes. In general, lipids commonly used in liposome typically includes dialiphatic chain lipids such as phospholipids, diglycerides, and dialiphatic glycolipids; single lipids such as sphingomyelin and glycosphingolipid; steroids such as cholesterol and derivatives thereof, and combinations thereof. Examples of phospholipids include, but are not limited to, phosphatidylethanolamine (PE), phosphatidylglycerol (PG), phosphatidylcholine (PC), 1,2-dilauroyl-sn-glycero-3-phosphocholine (DLPC), 1,2-dimyristoyl-sn-glycero-3-phosphocholine (DMPC), 1,2-dipalmitoyl-sn-glycero-3-phosphocholine (DPPC), 1-palmitoyl-2stearoyl-sn-glycero-3-phosphocholine (PSPC), 1-palmitoyl 2-oleoyl-sn-glycero-3-phosphatidylcholine (POPC), 1,2-distearoyl-sn-glycero-3-phosphocholine (DSPC), 1,2-diolcoyl-sn-glycero-3-phosphocholine (DOPC), hydrogenated soy phosphatidylcholine (HSPC), 1,2-dimyristoyl-sn-glycero-3-phospho-(1′-rac-glycerol) (sodium salt) (DMPG), 1,2-dipalmitoyl-sn-glycero-3-phospho-(1′-rac-glycerol) (sodium salt) (DPPG), 1-palmitoyl-2-stearoyl-sn-glycero-3-phospho-(1′-rac-glycerol) (sodium salt) (PSPG), 1,2-distearoyl-sn-glycero-3-phospho-(1′-rac glycerol) (sodium salt) (DSPG), 1,2-diolcoyl-sn-glycero-3-phospho-(1′-rac-glycerol) (DOPG), 1,2-dimyristoyl-sn-glycero-3-phospho-L-seine (sodium salt) (DMPS), 1,2-dipalmitoyl-sn-glycero-3-phospho-L-serine (sodium salt) (DPPS), 1,2-distearoyl-sn-glycero-3-phospho-L-serine (sodium salt) (DSPS), 1,2-diolcoyl-sn-glycero-3-phospho-L-serine (DOPS), 1,2-dimyristoyl-sn-glycero-3-phosphate (sodium salt) (DMPA), 1,2-dipalmitoyl-sn-glycero-3-phosphate (sodium salt) (DPPA), 1,2-distearoyl-sn-glycero-3-phosphate (sodium salt) (DSPA), 1,2-diolcoyl-sn-glycero-3-phosphate (sodium salt) (DOPA), 1,2-dipalmitoyl-9n-glycero-3-phosphoethanolamine (DPPE), 1-palmitoyl-2-oleoyl-sn-glycero-3-phosphoethanolamine (POPE), 1,2-distearoyl-sn-glycero-3-phosphoethanolamine (DSPE), 1,2 dioleoyl-sn-glycero-3-phosphoethanolamine (DOPE), 1,2-dipalmitoyl-sn-glycero-3-phospho-(1′-myo-inositol) (ammonium salt) (DPPI), 1,2-distearoyl-sn-glycero-3-phosphoinositol (ammonium salt) (DSPI), 1,2-diolcoyl-sn-glycero-3-phospho-(1-myo-inositol) (ammonium salt) (DOPI), cardiolipin, L-alpha-phosphatidylcholine (EPC), 1,2-diolcoyl-sn-glycero-3-cthylphosphocholine (18:1 EPC), L-alpha-phosphatidylethanolamine (EPE), dimethyldioctadecylammonium (DDAB), 1,2-dioleoyl-3-trimethylammonium-propane (DOTAP), 1,2-di-O-octadecenyl-3-trimethylammonium propane (DOTMA), and 3beta-[N—(N′,N′-dimethylaminoethane)-carbamoyl]cholesterol hydrochloride.

The suitable lipid may be a lipid mixture of one or more of the foregoing lipids, or mixtures of one or more of the foregoing lipids with one or more other lipids not listed above, membrane stabilizers or antioxidants.

The mole percentage of the lipid in the bilayer membrane may be equal or less than about 50, 45, 40, 35, 30, 25, 20, 15, 10, 5 or any value or range of values therebetween (e.g., about 5-50%, about 5-45%, about 5-40%, about 5-35%, about 5-30%, about 5-25%, about 5-20%, about 5-15%, or about 5-10%).

The lipid of the bilayer membrane may be a mixture of a first phospholipid and a second phospholipid. The first phospholipid may be selected from the group consisting essentially of PC, HSPC, DOPC, POPC, DSPC, DPPC, DMPC, PSPC and combinations thereof, and the second phospholipid is selected from the group consisting essentially of a PE, PG, DOPE, PEG-DSPE, DPPG, DOPG, DOTAP, DOTMA, DDAB and combination thereof. In other embodiments, the mole percentage of the first phospholipid in the bilayer membrane is about 50, 45, 40, 35, 30, 25, 20, 15, 10 or any value or range of values therebetween (e.g., about 5-50%, about 5-45%, about 5-40%, about 5-35%, about 5-30%, about 5-25%, about 5-20%, about 5-15%, or about 5-10%) and the mole percentage of the second phospholipid in the bilayer membrane is between 0.1 to about 15, 14, 13, 12, 11, 10, 9, 8, 7 or any value or range of values therebetween (e.g., about 0.1-15%, about 0.1-10%, about 0.5-15%, about 0.5-10% or about 0.5-7%). In an exemplary embodiment, the first phospholipid (DSPC) and the second phospholipid (DOPE, DOPG or DDAB) may be at a molar ratio of 4:1 to 6:1.

In an exemplary embodiment, the bilayer membrane of the liposome includes less than about 55 mole percentage of steroids, preferably cholesterol. The mole percentage of steroid (such as cholesterol) in the bilayer membrane may be about 15-55%, about 20-55%, about 25-55%, about 15-50%, about 20-50%, about 25-50%, about 15-45%, about 20-45%, about 25-45%, about 15-40%, about 20-40% or about 25-40%. The mole percentage of the phospholipid and cholesterol in the bilayer membrane may be about 25-50%:15-55%, 25-50%:20-55% or 25-50%:15-50%. The phospholipid(s) and cholesterol may be at a molar ratio of 1:1 to 3:1. The mole percentage ratio of the first phospholipid, the second phospholipid and cholesterol in the bilayer membrane may be about 25-50%:0.1-15%:15-55%, 5-50%:0.1%-15%:10-40%, or 25-50%:0.5-10%:5-20%.

The liposome encapsulating a trapping agent can be prepared by any of the techniques now known or subsequently developed. For example, the MLV liposomes can be directly formed by a hydrated lipid film, spray-dried powder or lyophilized cake of selected lipid compositions with trapping agent; the SUV liposomes and LUV liposomes can be sized from MLV liposomes by sonication, homogenization, microfluidization or extrusion.

In yet another embodiment, the compound including the retinoic acid conjugated with the carbohydrate, or the pharmaceutically acceptable salt thereof, and the first and second pharmaceutical compositions can be used to inhibit infection or replication of a virus. The virus may be an RNA virus. The RNA virus can include coronavirus, human immunodeficiency virus (HIV), hepatitis C virus (HCV), influenza virus, or any combination thereof. The coronavirus may include severe acute respiratory syndrome coronavirus (SARS-CoV), severe acute respiratory syndrome coronavirus 2 (SARS-COV-2), Middle East respiratory syndrome coronavirus (MERS-COV), human coronavirus 229E (HcoV-229E), human coronavirus OC43 (HCoV-OC43), human coronavirus NL63 (HCoV-NL63), human coronavirus HKU (HCoV-HKU1), or any combination thereof. In an exemplary embodiment, the coronavirus is SARS-COV-2 that causes COVID-19.

In further another embodiment, the compound including the retinoic acid conjugated with the carbohydrate, the pharmaceutically acceptable salt thereof, and the first and second pharmaceutical compositions can be used to treat a cancer. The cancer may include leukemias, lymphomas, carcinomas, or sarcomas. In exemplary embodiments, the cancer may be lung cancer, ovarian cancer, breast cancer, liver cancer, pancreatic cancer, or cholangiocarcinoma.

In certain embodiments, the compound and the metal ion in the first pharmaceutical composition may be administered separately, simultaneously, or sequentially. The retinoic acid and the carbohydrate in the second pharmaceutical composition may be administered separately, simultaneously, or sequentially. All or at the least two of the retinoic acid, the carbohydrate and the metal ion in the second pharmaceutical composition may be administered separately, simultaneously, or sequentially. In an exemplary embodiment, the metal ion and the carbohydrate are administered simultaneously, followed by the retinoic acid.

In exemplary embodiments, time intervals between administrations sequentially may be from 1 to 30 minutes, from 30 to 60 minutes, from 60 to 90 minutes, from 90 to 120 minutes, from 2 to 3 hours, from 3 to 12 hours or from 12 to 24 hours.

In certain embodiments, the compound including the retinoic acid conjugated with the carbohydrate, the pharmaceutically acceptable salt thereof, and the first and second pharmaceutical compositions each can be prepared in dimethyl sulfoxide (DMSO), ethanol, buffer or water for administration. The dosage and the frequency of administration thereof may vary depending on the following factors: the severity of the viral infection (e.g., coronavirus infection), or illness (e.g., cancer) to be treated and the weight, age, physical condition and response of the subject to be treated. The daily dosage of the aforesaid treating agents may be administered in a single dose or in several doses.

In certain embodiments, the compound including the retinoic acid conjugated with the carbohydrate, the pharmaceutically acceptable salt thereof, and the first and second pharmaceutical compositions each are administered by an oral, intravenous, intramuscular, subcutaneous, intraperitoneal, intranasal or topical route.

### EXAMPLES

The present invention will be further described by way of the following examples. However, it should be understood that the following examples are intended solely for the purpose of illustration and should not be construed as limiting the present invention in practice.

### MATERIALS AND METHODS

### A. Preparation of the Novel Retinoic Acid Compound, Galactose-Modified Isotretinoin, Represented by Formula (Ia) Based on the Synthesis Scheme Below:

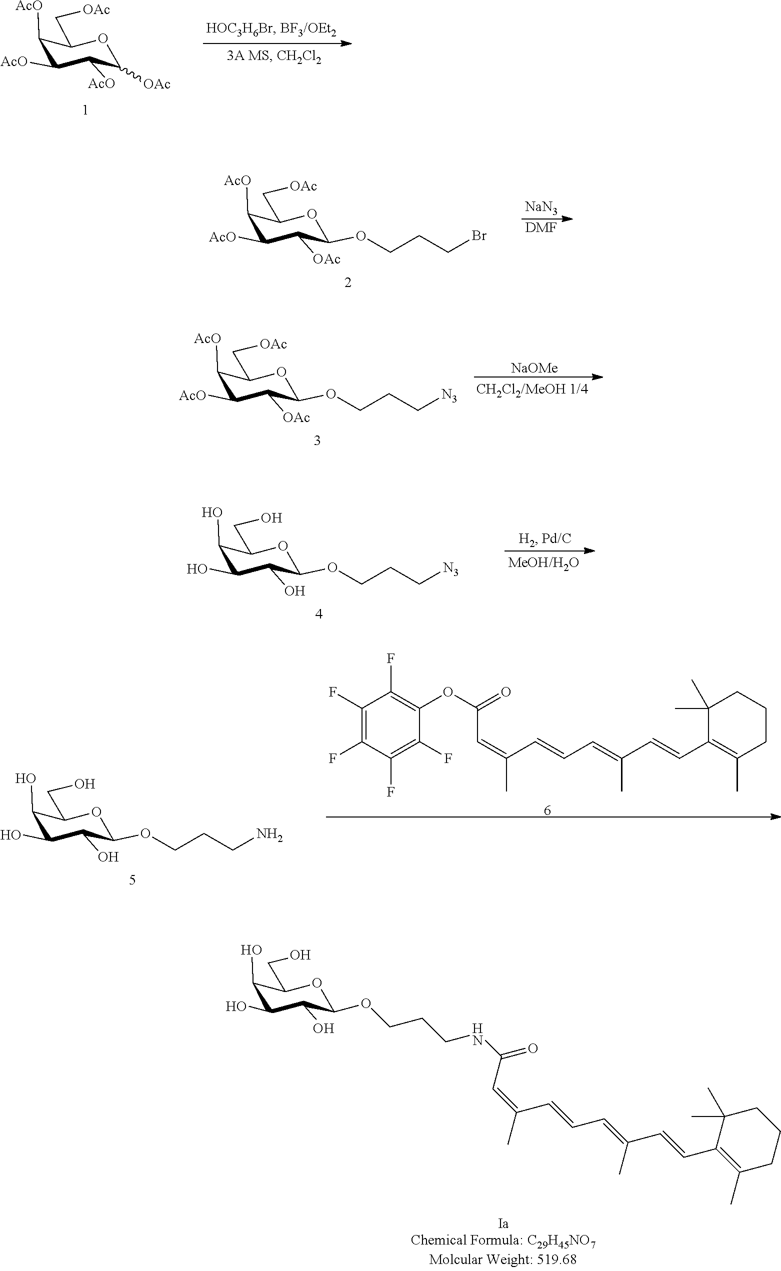

### I. General Experimental Methods

All reagents were commercial and were used without further purification. Yields refer to purified and spectroscopically pure compounds. Thin layer chromatography (TLC) was performed using Merck TLC Aluminum sheets silica gel 60 F254 plates and visualized by fluorescence quenching under UV light. Flash chromatography was performed using silica gel (Chromatorex, MB 70-40/75, 40-75 μm) purchased by Fuji Silysia Chemicals. NMR spectra were recorded on a Varian-400MR operating at 400 MHz for 1H. Chemical shifts are reported in ppm with the solvent resonance as the internal standard. Data is reported as follows: s=singlet, br=broad, d=doublet, t=triplet, q=quartet, m=multiplet, dd=doublet of doublets; coupling constants in Hz; integration. The purities were recorded on Waters e2695 separations Module/2998 PDA Detector HPLC system (Column: XBridge C18, 5 μm, 4.6 mm (ID)×150 mm (L), Eluent: the mixture of mobile phase A and B, mobile phase A: 100% acetonitrile; mobile phase B: pure water containing 0.1% formic acid and 10 mM NH₄OAc, Flow rate: 0.5 mL/min. detection: UV, 254 nm).

### II. Synthesis of the compound (2R,3S,4S,5R,6R)-2-(acetoxymethyl)-6-(3-bromopropoxy)tetrahydro-2H-pyran-3,4,5-triyl triacetate (2)

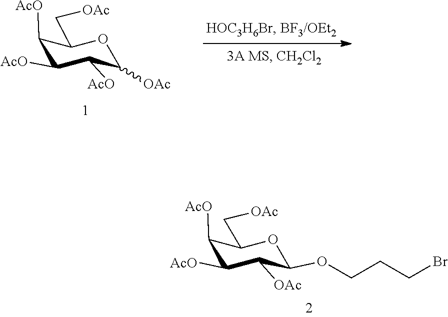

A solution of galactose pentaacetate (compound (1), 5.0 g, 13 mmol), 3-bromo-1-propanol (1.73 mL, 19.0 mmol) and fresh dried molecular sieve in anhydrous dichloromethane (50 mL) was added with boron trifluoride-diethyl etherate (8.04 mL, 64.0 mmol) at 0° C. The mixture was stirred at room temperature for overnight. The solution was neutralized with triethyl amine, the molecular sieve was removed by passing through celite, and the reaction mixture was washed with water and brine. The organic layer was dried over anhydrous magnesium sulfate and evaporated to dryness. The crude was purified by silica-gel column chromatography (EtOAc:hexane=1:2) to give the desire product (2) (0.625 g, 10%) as a colorless oil. ¹H NMR (400 MHZ, CDCl₃): δ 5.40 (dd, J=3.4, 1.2 Hz, 1H), 5.22-5.16 (m, 1H), 5.04-5.01 (m, 1H), 4.48 (d, J=8 Hz, 1H), 4.21-4.11 (m, 2H), 4.03-3.98 (m, 1H), 3.92 (td, J=5.8, 0.8 Hz, 1H), 3.72-3.66 (m, 1H), 3.50-3.47 (m, 2H), 2.25-1.99 (m, 14H); LCMS (ESI) m/z calcd for C₁₇H₂₅BrO₁₀ 469.28; found, 491.04 [M+Na]⁺.

### III. Synthesis of the Compound (2R,3S,4S,5R,6R)-2-(acetoxymethyl)-6-(3-azidopropoxy)tetrahydro-2H-pyran 3,4,5-triyl triacetate (3)

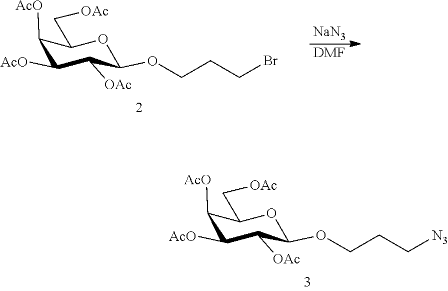

A solution of compound (2) (0.62 g, 1.3 mmol) in DMF (4 mL) was added with sodium azide (0.43 g, 6.6 mmol). The mixture was stirred at 100° C. for 12 h. The solution was concentrated to dryness. The crude was purified by silica-gel column chromatography (EtOAc:hexane=1:2) to give the desire product (3) (458 mg, 80%) as a colorless oil. ¹H NMR (400 MHZ, CDCl₃): δ 5.39 (dd, J=3.4, 0.8 Hz, 1H), 5.22-5.18 (m, 1H), 5.03-5.00 (m, 1H), 4.48 (d, J=8 Hz, 1H), 4.21-4.10 (m, 2H), 3.99-3.89 (m, 1H), 3.63-3.58 (m, 1H), 3.39-3.35 (m, 2H), 2.15 (s, 3H), 2.07 (s, 3H), 2.05 (s, 3H), 1.99 (s, 3H), 1.92-1.77 (m, 2H); LCMS (ESI) m/z calcd for C₁₇H₂₅N₃O₁₀ 431.40; found, 454.2 [M+Na]⁺.

### IV. Synthesis of the Compound (2R,3R,4S,5R,6R)-2-(3-azidopropoxy)-6-(hydroxymethyl)tetrahydro-2H-pyran-3,4,5-triol (4)

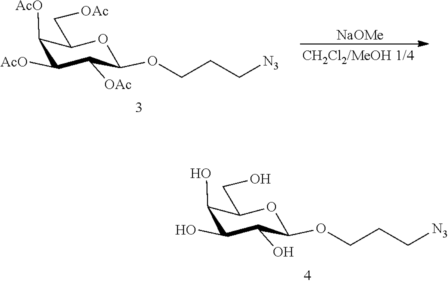

A solution of the compound (3) (0.45 g, 1 mmol) in dichloromethane (1.0 mL) and methanol (4.0 mL), and sodium methoxide (28 mg, 0.5 mmol) was added. The mixture was stirred at room temperature for 10 h. The solution was neutralized with Dowex 50WX8 and evaporated to dryness to give the desire product (4) which was used to next step without further purification. LCMS (ESI) m/z calcd for C₉H₁₇N₃O₆ 263.25; found, 286.1 [M+Na]⁺.

### V. Synthesis of the Compound (2R,3R,4S,5R,6R)-2-(3-aminopropoxy)-6-(hydroxymethyl)tetrahydro-2H-pyran-3,4,5-triol (5)

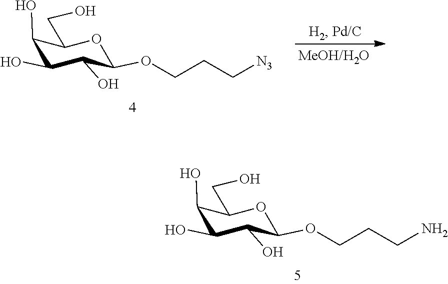

The crude compound (4) in methanol (10.5 mL) was treated with Pd/C (11 mg) and acetic acid (6.3 mg) under H₂ atmosphere. The solution was filtered through a pad of celite, and the filtrate was then concentrated under reduced pressure to afford (2R,3R,4S,5R,6R)-2-(3-aminopropoxy)-6-(hydroxymethyl)tetrahydro-2H-pyran-3,4,5-triol (5) which was used to next step without further purification. LCMS (ESI) m/z calcd for C₉H₁₉NO₆ 237.25; found, 237.8 [M]⁺.

### VI. Synthesis of the Compound perfluorophenyl (2Z,4E,6E,8E)-3,7-dimethyl-9-(2,6,6-trimethylcyclohex-1-en-1-yl)nona-2,4,6,8-tetraenoatee (6)

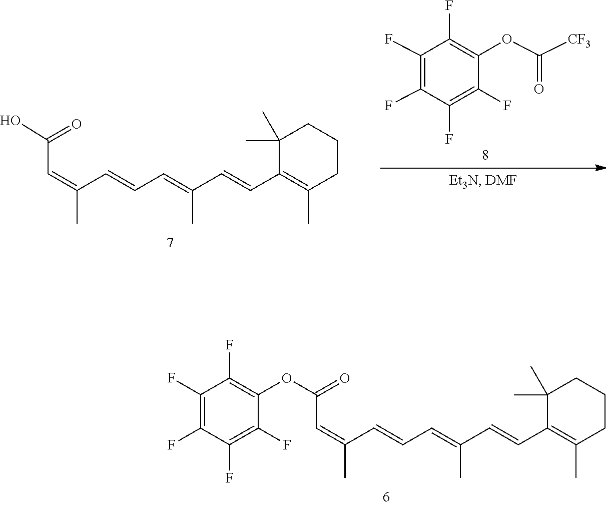 [SMILES: CC1=C(/C=C/C(C)=C/C=C/C(C)=C\C(=O)O)C(C)(C)CCC1 ; O=C(Oc1c(F)c(F)c(F)c(F)c1F)C(F)(F)F ; CC1=C(/C=C/C(C)=C/C=C/C(C)=C\C(=O)Oc2c(F)c(F)c(F)c(F)c2F)C(C)(C)CCC1]

A solution of retinoic acid (compound (7), 0.10 g, 0.33 mmol) and triethyl amine (0.093 mL, 0.67 mmol) in anhydrous DMF (1.0 mL) was added with pentafluorophenyl trifluoroacetate (compound (8), 0.081 mL, 0.47 mmol) at 0° C. The solution was stirred at room temperature for 2 hours. The reaction mixture is diluted with EtOAc and washed with 0.1 N aqueous HCl, followed by aqueous NaHCO₃, and brine. The organic layer was dried over anhydrous magnesium sulfate, and concentrated to dryness to give the desired product (6) without further purification.

### VII. Synthesis of the Target Compound (2Z,4E,6E,8E)-3,7-dimethyl-N-(3-(((2R,3R,4S,5R,6R)-3,4,5-trihydroxy-6-(hydroxymethyl)tetrahydro-2H-pyran-2-yl)oxy)propyl)-9-(2,6,6-trimethylcyclohex-1-en-1-yl) nona-2,4,6,8-tetraenamide (1a)

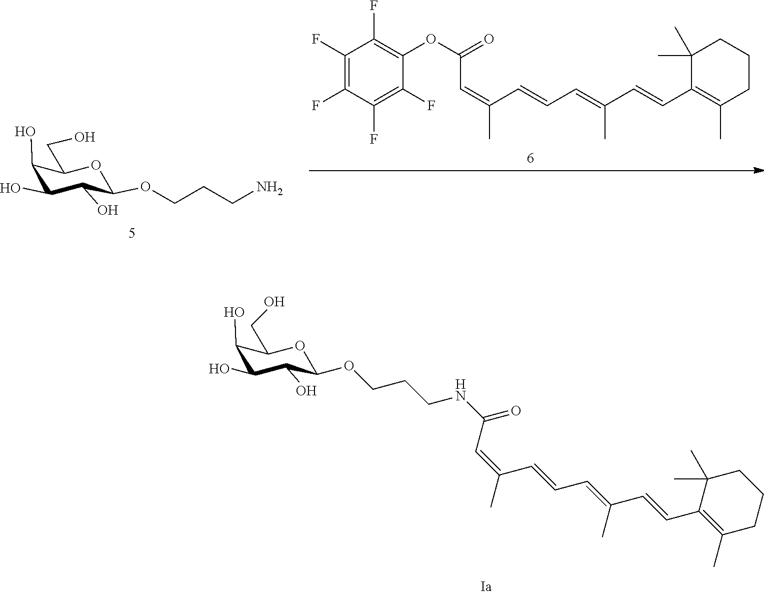

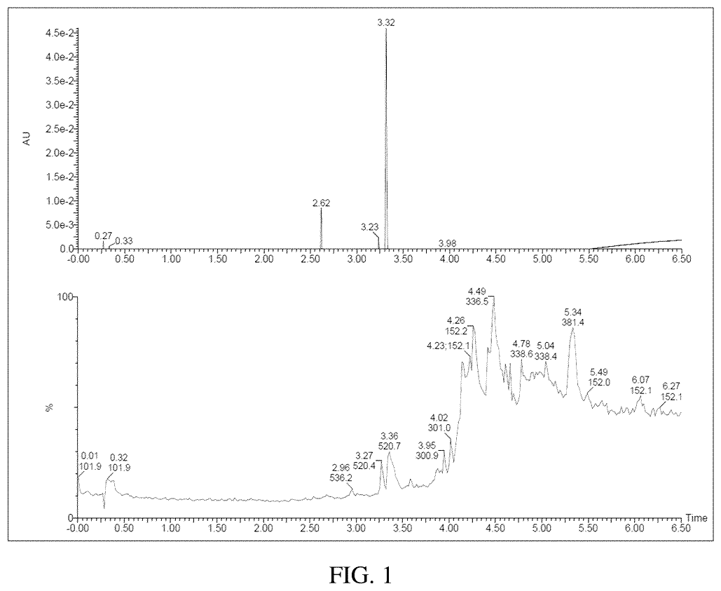

A solution of crude compound (5) (30 mg, 0.13 mmol) and activated ester (6) (150 mg, 0.33 mmol) in DMF (1.0 mL) was added with triethylamine (0.049 mL, 0.33 mmol) at room temperature. The mixture was stirred for overnight. The solution was concentrated to dryness. The crude was purified by silica-gel column chromatography (methanol:dichloromethane=1:9). LCMS (ESI) m/z calcd for C₂₉H₄₅NO₇ 519.68; found, 520.5 [M+H]⁺. See FIG. 1.

### B. Preparation of SARS-COV-2 PLPro

In order to determine whether the claimed compound and pharmaceutical compositions could inhibit SARS-COV-2, their in vitro inhibitory effects on the activity of SARS-COV-2 papain-like protease (PLpro) was first assessed.

The codon-optimized gene sequence encoding wild-type SARS-COV-2 PLpro was synthesized by Biotools Co., Ltd. (New Taipei City, Taiwan) and sub-cloned into pET-21a (Novagen) vector using the NdI and XhoI restriction sites, while the His-tag coding region (-LEHHHHHH-) was retained at the C-terminus.

The vector with the inserted SARS-COV-2 PLPro gene was transformed into E. coli BL21 (DE3) strain (Yeastern Biotech Co., Ltd., New Taipei City, Taiwan) for overexpression of PLPro therein. Cultivation was performed in LB medium (containing 1% tryptone, 0.5% yeast extract, and 1% NaCl) supplemented with ampicillin (100 μg/mL) serving as an antibiotic marker. The resultant culture was initially incubated at 37° C. with being shaken at 200 rpm. At an optical density at 600 nm (OD₆₀₀) between 0.6 and 0.8, isopropyl β-D-1-thiogalactopyranoside (IPTG) was added to reach a final concentration of 0.4 mM to induce the expression of PLpro. Incubation continued at 18° C. and 200 rpm for 20 hours. The cells were harvested by centrifugation (5,000×g) and disrupted by sonication in a lysis buffer containing 50 mM sodium phosphate (pH 7.4), 1.0 mM DTT, 5% glycerol and 100 mM NaCl. The cell debris was then removed by centrifugation at 20,000×g for 50 minutes. The supernatant was loaded onto a 5 mL His-Trap HP column (GE Healthcare Life Sciences), and the protein therein was eluted, using a gradient of 0-500 mM imidazole in 50 mM sodium phosphate (pH 7.4) and 100 mM NaCl. Fractions containing His-tagged SARS-COV-2 PLpro were pooled and concentrated, using a Centricon membrane (10 K cutoff, GE Healthcare Life Sciences). His-tagged SARS-COV-2 PLpro was further purified by gel filtration chromatography, using Superdex 75 gel filtration column (GE Healthcare Life Sciences) in a 50 mM sodium phosphate buffer (pH 7.4). The SARS-COV-2 PLpro concentration was determined by measuring the ultraviolet absorbance at 280 nm, using an extinction coefficient (&280) of 45270 M⁻¹ cm⁻¹.

### C. SARS-COV-2 PLPro Activity Assay

The enzymatic activity of SARS-COV-2 PLpro obtained above was measured by a colorimetry-based peptide cleavage assay, using the 6-mer peptide substrate, FRLKGG-para-nitroanilide (FG6-pNA) (purity 97% by HPLC; GL Biochem Ltd., Shanghai, China). In the cleavage assay, the 6-mer peptide substrate was cleaved at the Gly-pNA bond to release free pNA, which turned the color of the solution to yellow. The enzymatic activity was determined by continuously monitoring the absorbance at 405 nm (A405) using a 96-well microplate spectrophotometer (Epoch™ 2, Biotek) at 30° C.

Specifically, the cleavage assay was conducted in a 96-well microplate. Each of the wells of the microplate contained a 50 mM phosphate buffer (pH 7.4), and FG6-pNA was added into the respective well such that substrate solutions having various concentrations of FG6-pNA (0.1875 mM, 0.375 mM, 0.75 mM, 1.5 mM, 3.0 mM, 6.0 mM) were prepared. The assay mixture (180 μL in each well) was preincubated for 10 minutes for accurate temperature control, and the reaction was initiated by adding 20 μL of a SARS-COV-2 PLpro solution (1.75 μM) to the assay mixture. The SARS-COV-2 PLpro solution was prepared by admixing the SARS-COV-2 PLpro obtained above with a 50 mM sodium phosphate buffer (pH 7.4). The concentration of pNA released by proteolysis was calculated by measuring A₄₀₅, using an extinction coefficient (ε405) of 9800 M⁻¹ cm⁻¹ (A₄₀₅=9.8 at 1 mM).

The steady state enzyme kinetic parameters were obtained by fitting the initial velocity (V₀) data based on the Michaelis-Menten Equation, using the OriginPro 8.0 software (OriginLab Corporation, USA). All measurements were performed in triplicate. The data obtained are expressed as mean±standard deviation.

Results: the K_{M} and k_{cat} values are 2.50±0.03 mM and 0.85±0.01 s⁻¹, respectively. Therefore, it is verified that SARS-COV-2 PLPro having protease activity was successfully prepared according to the above manufacturing method, and could be used to perform the following SARS-COV-2 PLPro inhibition assay.

### D. Establishment of SARS-COV-2 Animal Model

Golden Syrian hamsters (aged 5-6 weeks old and having an average weight of about 100 g) were obtained from the National Laboratory Animal Center (Taipei, Taiwan). The hamsters were housed in an animal room under specific-pathogen-free (SPF) conditions commonly applied in the art. Furthermore, water and feed were provided ad libitum for all the hamsters. All the experiments involving the hamsters were consigned to and reviewed and approved by the Institutional Animal Care and Use Committee (IACUC) of Academia Sinica (Taiwan).

The hamsters were infected with SARS-COV-2 (obtained from P3 Lab, the Genomics Research Center, Academia Sinica; WuHan wild type) in phosphate buffered saline (PBS) through intranasal inoculation at 1×10⁴ plaque-forming units (PFU) on 12:00 PM so as to establish a SARS-COV-2 animal model. The establishment of the SARS-COV-2 animal model was confirmed (data not shown).

### Example 1

### SARS-COV-2 PLPro Inhibition Assay for In Vitro Evaluating Antiviral Effect of Novel Retinoic Acid Compound, Galactose-Modified Isotretinoin, Against SARS-COV-2

An enzyme inhibition assay was performed in a 96-well microplate. Each of the wells of the microplate contained a 50 mM phosphate buffer (pH 7.4). SARS-COV-2 PLPro obtained above (0.9 μM) was added into the respective well to form an enzyme solution. The enzyme solutions in the wells were divided into one control group (abbreviated as “Control”), one comparative group (abbreviated as “Comparative_1”) and one experimental group (abbreviated as “Experimental_1”). To the enzyme solution of the respective group, the corresponding inhibiting agent shown in Table 1 below was added to form a test mixture (with a total volume of 180 μL). Preincubation was conducted for 30 minutes.

TABLE 1

| Groups | Components |
|---|---|
| Control | PLpro |
| Comparative_1 | PLpro + 50 μM isotretinoin |
| Experimental_1 | PLpro + 50 μM Galactose-modified isotretinoin |

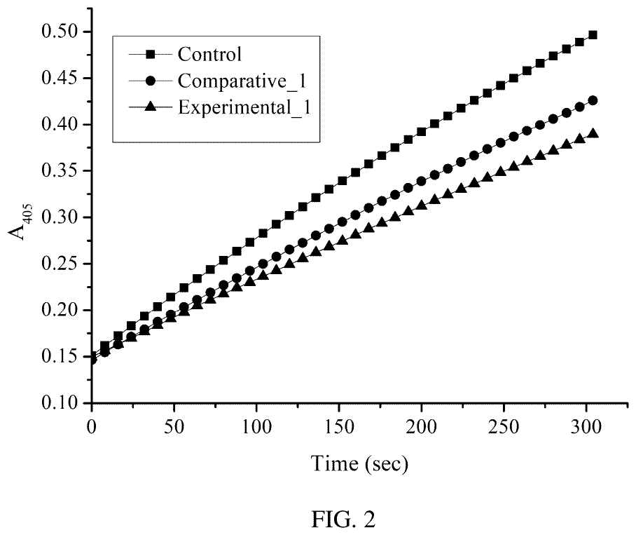

20 μL of FG6-pNA (1.2 mM) described above was added into the test mixture of the respective group to initiate the enzyme reaction. The enzyme reaction was allowed to proceed at 30° C. for 300 seconds. The enzymatic activity was determined by continuously monitoring the absorbance at 405 nm (A₄₀₅) using a 96-well microplate spectrophotometer (Epoch™ 2, Biotek). The reaction rate was calculated accordingly, wherein the reaction rate is the slope of the absorbance A₄₀₅ versus time (seconds) for a total reaction time of 300 seconds. The data are presented in FIG. 2.

The inhibition percentage was calculated using the following equation:

$$A = [ 1 - ( B / C ) ] \times 100 \qquad ( I )$$

$$where A = inhibition percentage$$

$$B = reaction rate of respective group$$

$$C = reaction rate of control group$$

The data obtained are expressed as mean±standard deviation.

Results: the inhibition percentages of different treatment groups are shown in Table 2 below.

TABLE 2

| Groups | Inhibition percentage (%) |
|---|---|
| Control: | 0 |
| PLpro | |
| Comparative_1: | 20.90 ± 1.24 |
| PLpro + isotretinoin | |
| Experimental_1: | 34.21 ± 0.17 |
| PLpro + Galactose-modified isotretinoin | |

As shown in FIG. 2 and Table 2, the inhibition rate of Experimental_1 was significantly higher than that of Comparative_1, indicating that the galactose-modified isotretinoin was more effective than isotretinoin only in inhibiting the enzymatic activity of PLpro. This result suggests that galactose-modified isotretinoin have a better inhibitory effect on the activity of papain-like protease.

In addition, it provides important insights into the biochemical properties of the coronaviral PLpro family and paves the way for promising therapeutic strategies against SARS-COV-2.

### Example 2

### Evaluation for In Vivo Antiviral Effect of Novel Retinoic Acid Compound, Galactose-Modified Isotretinoin, Against SARS-COV-2 in SARS-COV-2 Animal Model

Since the novel retinoic acid compound, galactose-modified isotretinoin, was proven to have in vitro inhibitory effect against SARS-COV-2, an in vivo animal test for evaluating antiviral effect of the novel retinoic acid compound against SARS-COV-2 was further assessed.

The infected hamsters obtained as mentioned above were divided into the following four groups (n=5 per group): a control group (abbreviated as “Control”), two comparative groups (respectively abbreviated as “Comparative_1” and “Comparative_2”) and one experimental group (abbreviated as “Experimental_1”). The therapeutic agents respectively used for these groups are listed in Table 3 below.

TABLE 3

| Groups | Therapeutic agents |
|---|---|
| Control | Buffer containing 40% ethanol, 40% |
| | Span ® 80 (sorbitan monooleate) (Sigma- |
| | Aldrich), and 20% peanut oil |
| Comparative_1 | Isotretinoin |
| Comparative_2 | Liposome-encapsulated isotretinoin |
| Experimental_1 | Galactose-modified isotretinoin |

Specifically, for each hamster in Experimental_1, the new retinoic acid compound was administered intranasally at 8:00 AM on the infection day (i.e., 4 hours before the SARS-CoV-2 infection, day 1) at a dose of 0.35 mg/kg and at 8:00 PM on the infection day at a dose of 0.35 mg/kg. On the next two days (day 2 and 3), the new retinoic acid compound was administered twice daily at 8:00 AM and 8:00 PM through nasal administration at a dose of 0.35 mg/kg each.

For each hamster in the comparative groups, isotretinoin in Comparative_1 or liposome-encapsulated isotretinoin in Comparative_2 was administered intranasally at 8:00 AM on the day of infection (i.e., 4 hours before the SARS-COV-2 infection) at a dose of 0.35 mg/kg and at 8:00 PM on the day of infection at a dose of 0.35 mg/kg. Isotretinoin in Comparative_1 or liposome-encapsulated isotretinoin in Comparative_2 was administered intranasally twice daily at a dose of 0.35 mg/kg at 8:00 AM and 8:00 PM on the next two days after the day of infection. Isotretinoin encapsulated in the liposome in Comparative_2 was prepared by Taipei Medical University (Taiwan) by techniques commonly used in the art and described in detail above. Since the main technical feature of the present disclosure lies in the novel retinoic acid compound, galactose-modified isotretinoin, the details of liposome are omitted here for the sake of brevity.

For each hamster in the control group, the buffer was administered through nasal administration at 8:00 AM on the day of infection (i.e., 4 hours before the SARS-COV-2 infection) in a volume of 100 μL and at 8:00 PM on the infection day in a volume of 100 μL. The buffer was administered twice daily through nasal administration in a volume of 100 μL at 8:00 AM and 8:00 PM on the two days after the day of infection.

After the 3-day treatment, the hamsters were sacrificed, and the lungs thereof were collected for live viral load measurement by TCID₅₀ assay in Vero E6 cells. The virus titer was determined in terms of the 50% tissue culture infectious dose (TCID₅₀) using the Reed-Muench method. All the experiments with SARS-COV-2 were conducted in the biosafety level 3 (BSL-3) laboratory and were approved by Academia Sinica (Taipei, Taiwan).

The experimental data were analyzed by Tukey's test so as to evaluate the differences between the groups. Statistical significance is indicated by p<0.05.

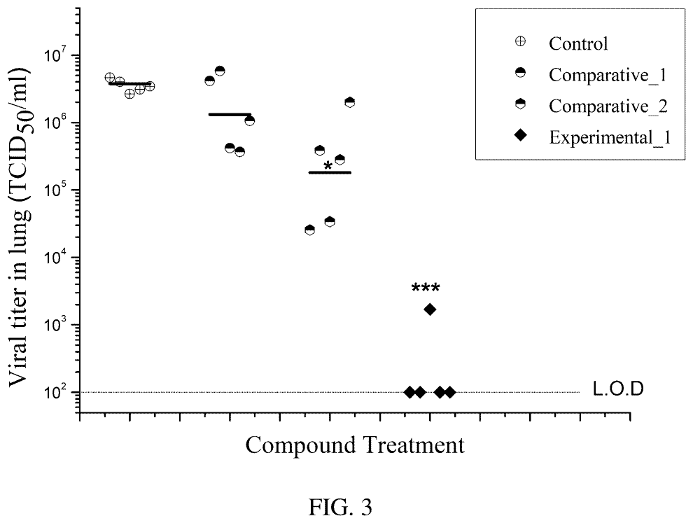

Results: referring to FIG. 3, TCID₅₀ of Experimental_1 was significantly lower than each of Comparative_1 and Comparative_2, revealing that the novel retinoic acid compound, galactose-modified isotretinoin, has higher in vivo efficacy against SARS-COV-2 compared to only isotretinoin or only liposome-encapsulated isotretinoin.

### Example 3

### Evaluation for In Vitro Cytotoxicity Effect of Novel Retinoic Acid Compound, Galactose-Modified Isotretinoin, on Different Cancer Cell Lines

In the present example, seven cell lines were used, and are respectively AsPC-1, MDA-MB-231, HCT-116, Huh-7, SKOV-3, A549 and H460 cancer cell lines. Each cell line was cultured in indicated growth medium with 10% FBS and were maintained in humidified incubator at 37° C. containing 5% CO₂.

One day before treatment, cells at log-phase of growth were harvested, counted, and seeded in a 96-well plate at a density of 1×10⁴ cells/100 μL/well. After cultivation overnight, culture media of each well were removed gently and fresh media were added (200 μL/well) subsequently.

The test article (TA) was the novel retinoic acid compound (abbreviated as “New Compound” in the present example) was freshly prepared on the day of treatment with 100% DMSO into a 96 mM stock solution. Isotretinoin was serially diluted 2-fold using 100% DMSO to obtain different concentrations of isotretinoin from 7.5 mM to 96 mM. Treat cells by adding 2.02 μL of the indicated concentrations of isotretinoin to each well to give a final concentration range of isotretinoin from 7.5 μM to 960 μM and keep all wells containing 1% DMSO, including the DMSO control. All components were gently mixed and incubated for 24 hr.

Cells were treated by adding 2.02 μL of indicated concentrations of New Compound A or DMSO to each well, and then mixed gently and incubated for 24 hours.

At the day of cell viability, culture medium of each well was replaced by fresh-prepared Alamar blue dye (10% v/v) and incubated at 37° C. for 2 to 3 hours. Spectrophotometric absorbances were recorded at wavelengths of 570 nm and 600 nm.

The percent of cell viability were calculated using the following formula:

$$\% viability = ( [ {A}_{570} / {A}_{600} ratio ] of TA / [ {A}_{570} / {A}_{600} ratio ] of DMSO ) * 100 \%$$

$$where TA = test article$$

$${A}_{570} = absorbance at 570 nm$$

$${A}_{600} = absorbance at 600 nm$$

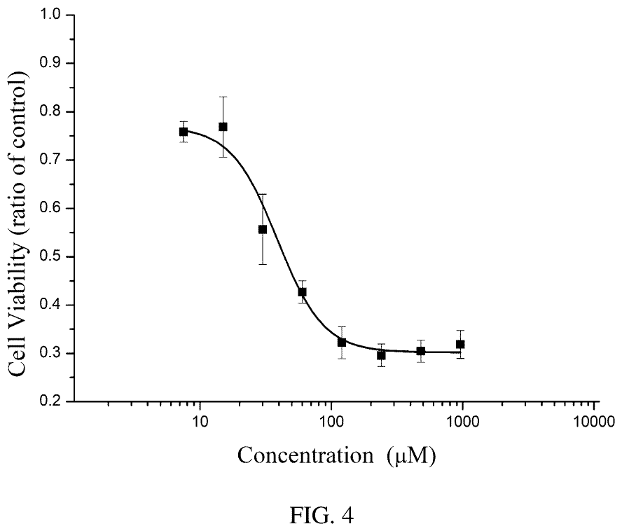

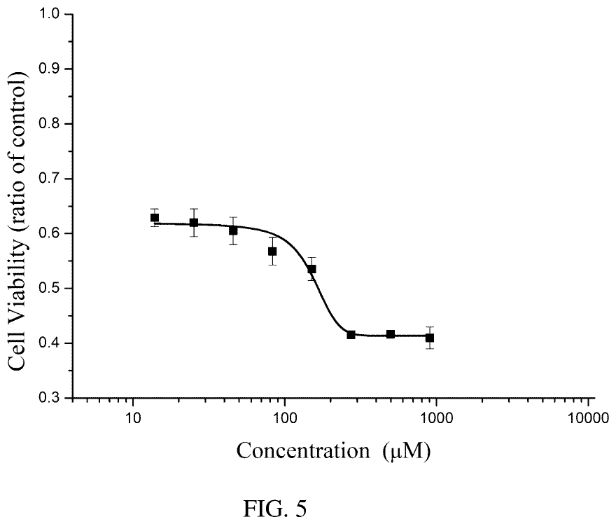

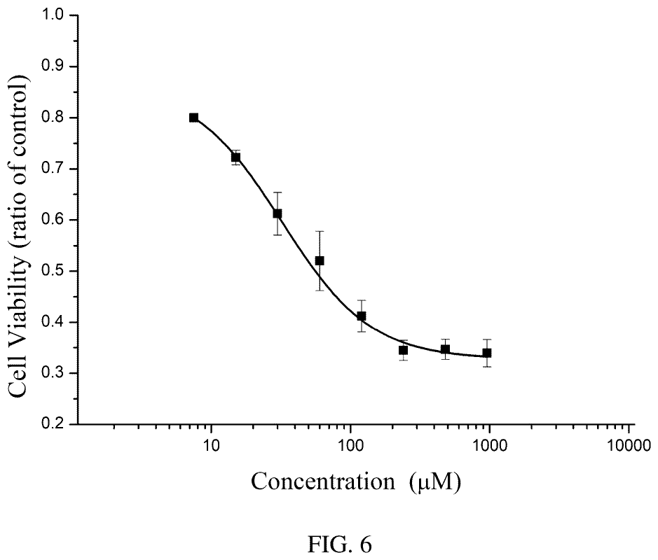

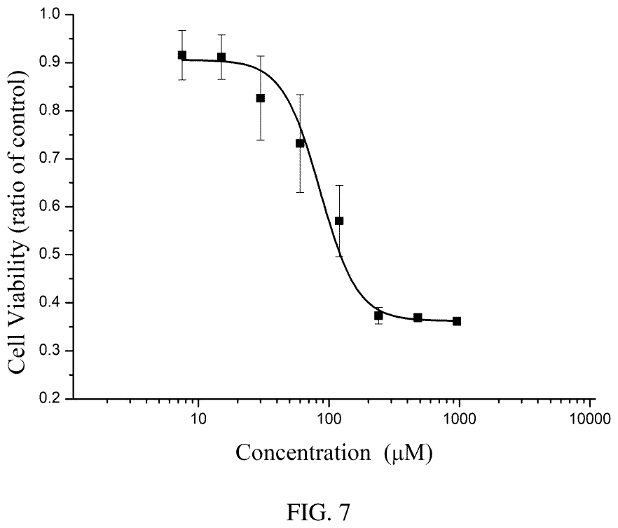

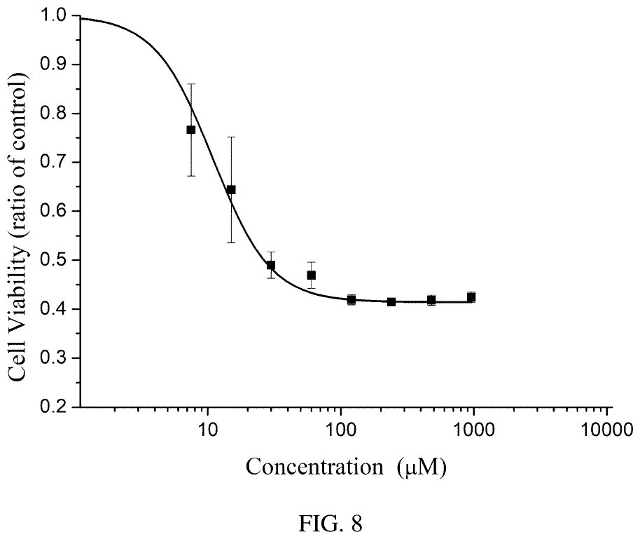

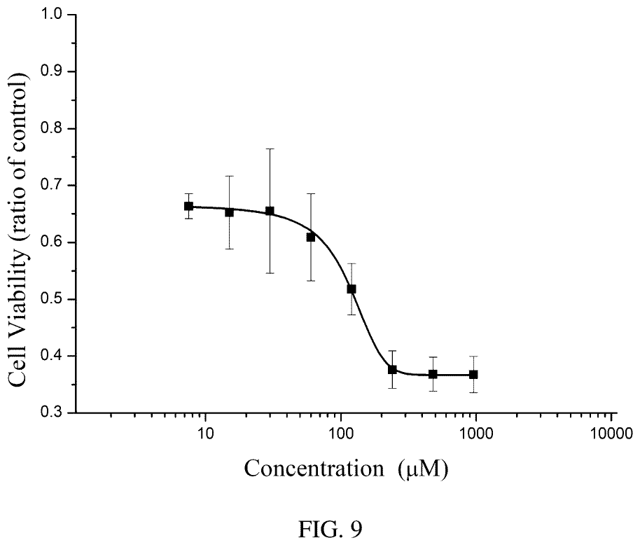

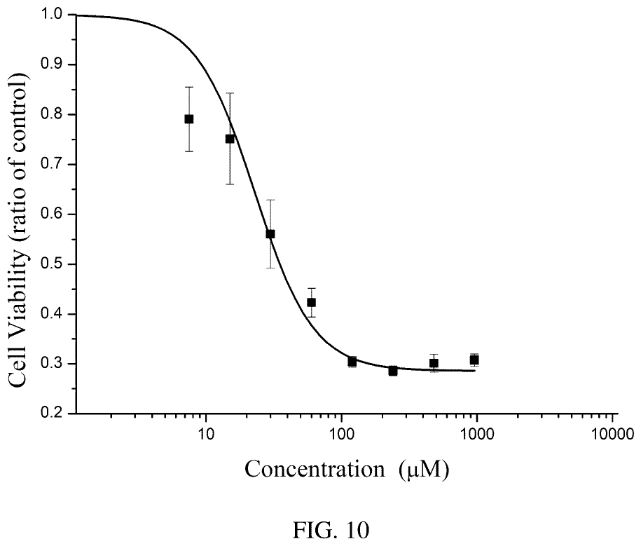

Results: referring to FIGS. 4-10, they shows New Compound can significantly reduce the cell viability of the all seven cancer cell lines, revealing that the novel retinoic acid compound, galactose-modified isotretinoin, may be a potential anticancer drug for effectively treating various cancers, such as lung cancer, ovarian cancer, breast cancer, pancreatic cancer, colon cancer, and liver cancer. Showed the IC₅₀ in Table 4.

TABLE 4

| Tumor type | Cell line | IC₅₀ (μM) |
|---|---|---|
| Non-small Cell Lung Cancer | NCI-H460 | 22.95 |
| Non-small Cell Lung Cancer | A549 | 21.88 |
| Liver Cancer | Huh-7 | 77.46 |
| Pancreatic Cancer | AsPC-1 | 23.00 |
| Colon Cancer | HCT-116 | 123.44 |
| Ovarian Cancer | SK-OV-3 | 13.82 |
| Breast Cancer | MDA-MB-231 | 115.88 |

### Example 4

### SARS-COV-2 PLPro Inhibition Assay for In Vitro Evaluating Antiviral Effect of the Second Pharmaceutical Compositions Against SARS-COV-2

An enzyme inhibition assay was performed in a 96-well microplate. Each of the wells of the microplate contained a 50 mM phosphate buffer (pH 7.4). SARS-COV-2 PLPro obtained as mentioned above was added into the respective well to form a final concentration of 0.9 μM of enzyme solution. The enzyme solutions in the wells were divided into one control group (abbreviated as “Control”), four comparative groups (respectively abbreviated as “Comparative_1,” “Comparative_2,” “Comparative_3” and “Comparative_4”), and two experiment groups (respectively abbreviated as “Experimental_1” and “Experimental_2”). To the enzyme solution of the respective group, the corresponding inhibiting agent shown in Table 5 below was added to form a test mixture (with a total volume of 180 μL). Preincubation was conducted for 30 minutes.

TABLE 5

| Groups | Components |
|---|---|
| Control | PLpro |
| Comparative_1 | PLpro + mixture of oligosaccharides [(β-1,3/1,6- |
| | glucan oligosaccharides) (25 μM) + raffinose |
| | (25 μM) + stachyose (25 μM) + verbascose |
| | (25 μM)] |
| Comparative_2 | PLpro + Mg²⁺ (0.9 μM) |
| Comparative_3 | PLpro + Zn²⁺ (0.09 μM) |
| Comparative_4 | PLpro + isotretinoin (50 μM) |
| Experimental_1 | PLpro + isotretinoin (50 μM) + mixture of oligosac- |
| | charides [(β-1,3/1,6-glucan oligosaccharides) |
| | (25 μM) + raffinose (25 μM) + stachyose |
| | (25 μM) + verbascose (25 μM)] |
| Experimental_2 | PLpro + isotretinoin (50 μM) + mixture of oligosac- |
| | charides [(β-1,3/1,6-glucan oligosaccharides) |
| | (25 μM) + raffinose (25 μM) + stachyose |
| | (25 μM) + verbascose (25 μM)] + Mg²⁺ |
| | (0.9 μM) + Zn²⁺ (0.09 μM)) |

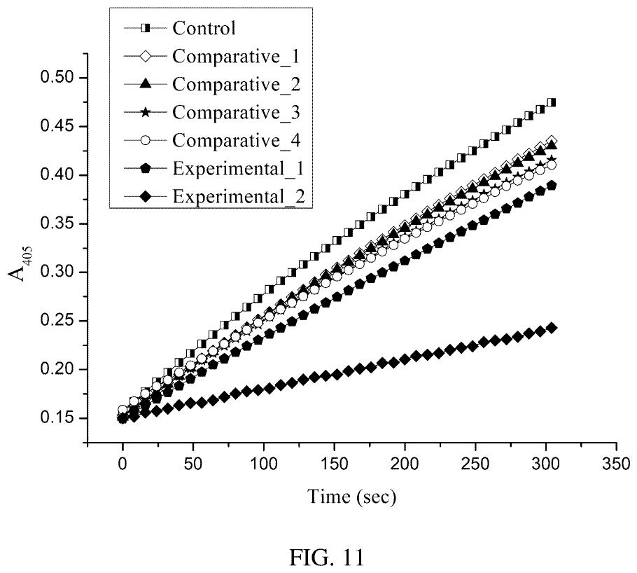

20 μL of FG6-pNA (1.2 mM) described above was added into the test mixture of the respective group to initiate the enzyme reaction. The enzyme reaction was allowed to proceed at 30° C. for 300 seconds. The enzymatic activity was determined by continuously monitoring the absorbance at 405 nm (A405) using a 96-well microplate spectrophotometer (Epoch™ 2, Biotek). The reaction rate was calculated accordingly, wherein the reaction rate is the slope of the absorbance A₄₀₅ versus time (seconds) for a total reaction time of 300 seconds). The data are presented in FIG. 11. The inhibition percentage was calculated as described in Example 3.

Results: the inhibition percentages of different treatment groups are shown in Table 6 below.

TABLE 6

| Group | Inhibition percentage (%) |
|---|---|
| Control: | 0 |
| PLpro | |
| Comparative_1: | 12.43 ± 0.37 |
| PLpro + oligosaccharides | |
| Comparative_2: | 14.21 ± 0.15 |
| PLpro + Mg²⁺ | |
| Comparative_3: | 19.22 ± 0.21 |
| PLpro + Zn²⁺ | |
| Comparative_4: | 20.90 ± 1.24 |
| PLpro + isotretinoin | |
| Experimental_1: | 26.61 ± 0.49 |
| PLpro + isotretinoin + oligosaccharides | |
| Experimental_2: | 72.16 ± 0.92 |
| PLpro + isotretinoin + | |
| oligosaccharides + Mg²⁺ + Zn²⁺ | |

As shown in FIG. 11 and Table 6, the inhibition effect of each group in the experimental groups was significantly higher than that of each group in the comparative groups, indicating that the two experimental groups: (1) isotretinoin and oligosaccharides, and (2) isotretinoin, oligosaccharides, Zn²⁺ and Mg²⁺, were more effective than the individual component of isotretinoin, oligosaccharides, Zn²⁺, or Mg²⁺ only. These results suggest that isotretinoin, oligosaccharides and divalent metal ions have a synergistic inhibitory effect on the activity of papain-like protease. In addition, it provides important insights into the biochemical properties of the coronaviral PLpro family and paves the way for promising therapeutic strategies against SARS-COV-2.

### Example 5

### Evaluation for In Vivo Treatment Effect for Combination of Isotretinoin, Oligosaccharides or Divalent Metal Ions Against SARS-COV-2 in SARS-COV-2 Animal Model

The infected hamsters obtained in part A of this case were divided into the following six groups (n=5 per group): a control group, three experimental groups (i.e. Experimental Groups 1, 2, and 3), and four comparative groups (i.e. Comparative Groups 1, 2, 3 and 4). The treatment agent respectively used for these groups are listed in Table 7 below.

TABLE 7

| Groups | Therapeutic agents |
|---|---|
| Control group | Buffer containing 40% ethanol, 40% Span ® 80 |
| | (sorbitan monooleate) (Sigma-Aldrich), and |
| | 20% peanut oil |
| Comparative_1 | Isotretinoin |
| Comparative_2 | Isotretinoin encapsulated in liposome |
| Comparative_3 | Combination of Zn²⁺, Mg²⁺ and K⁺ |
| | encapsulated in the liposome |
| Comparative_4 | Mixture of oligosaccharides encapsulated in |
| | the liposome |
| Experimental_1 | Isotretinoin encapsulated in liposome + |
| | mixture of oligosaccharides encapsulated in |
| | the liposome |
| Experimental_2 | Isotretinoin encapsulated in liposome + |
| | mixture of oligosaccharides encapsulated in |
| | liposome + |
| | combination of Zn²⁺, Mg²⁺ and K⁺ |
| | encapsulated in liposome |

The therapeutic agents encapsulated in liposomes were prepared by Taipei Medical University using liposomes by techniques commonly used in the art and described in detail above. Since the main technical feature of the present disclosure lies in the combination of retinoic acid and oligosaccharides with/without metal ions, the details of the liposomes are omitted here for the sake of brevity.

Specifically, for each hamster in the experimental groups, liposome-encapsulated isotretinoin was administered intranasally at 8:00 AM on the infection day (i.e., 4 hours before the SARS-COV-2 infection, day 1) at a dose of 0.35 mg/kg and at 8:00 PM on the infection day at a dose of 0.35 mg/kg. On the next two days (day 2 and 3), liposome-encapsulated isotretinoin was administered twice daily at 8:00 AM and 8:00 PM through nasal administration at a dose of 0.35 mg/kg each.

In addition that liposome-encapsulated isotretinoin was administered twice daily at 8:00 AM and 8:00 PM through nasal administration at a dose of 0.35 mg/kg, the mixture of oligosaccharides encapsulated in the liposome in a volume of 15 μL (i.e., Experimental_1), or the mixture of oligosaccharides (15 μL) and the combination of Zn²⁺ (100 μM), Mg²⁺ (200 μM) and K⁺ (200 μM) (15 μL), both of which are encapsulated in the liposome, in a total volume of 30 μL (i.e., Experiment_2) was given once daily through nasal administration from 7:30 PM to 8:00 PM (i.e., 0.5 hour to 1 hour before the administration of liposome-encapsulated isotretinoin at night) on the day of infection and two days thereafter in a volume of 15 μL each in Experimental_1 or 30 μL in Experimental_2.

For each hamster in the comparative groups, isotretinoin in Comparative_1 or liposome-encapsulated isotretinoin in Comparative_2 was administered intranasally at 8:00 AM on the day of infection (i.e., 4 hours before the SARS-COV-2 infection) at a dose of 0.35 mg/kg and at 8:00 PM on the day of infection at a dose of 0.35 mg/kg, isotretinoin in Comparative_1 or liposome-encapsulated isotretinoin in Comparative_2 was administered intranasally twice daily at a dose of 0.35 mg/kg at 8:00 AM and 8:00 PM on the next two days after the day of infection.

For each hamster, the combination of Zn²⁺ (100 μM), Mg²⁺ (200 μM) and K⁺ (200 μM) encapsulated in the liposome in Comparative_3 and the mixture of oligosaccharides encapsulated in the liposome in Comparative_4 was given once daily through nasal administration from 7:30 PM to 8:00 PM on the day of infection and two days thereafter in a volume of 15 μL each.

For each hamster in the control group, the buffer was administered through nasal administration at 8:00 AM on the day of infection (i.e., 4 hours before the SARS-COV-2 infection) in a volume of 100 μL and at 8:00 PM on the infection day in a volume of 100 μL. The buffer was administered twice daily through nasal administration in a volume of 100 μL at 8:00 AM and 8:00 PM on the two days after the day of infection.

After the 3-day treatment, the hamsters were sacrificed, and the lungs thereof were collected for live viral load measurement by TCID₅₀ assay in Vero E6 cells. The virus titer was determined in terms of the 50% tissue culture infectious dose (TCID₅₀) using the Reed-Muench method. All the experiments with SARS-COV-2 were conducted in the biosafety level 3 (BSL-3) laboratory and were approved by Academia Sinica (Taipei, Taiwan).

The experimental data were analyzed by Tukey's test so as to evaluate the differences between the groups. Statistical significance is indicated by p<0.05.

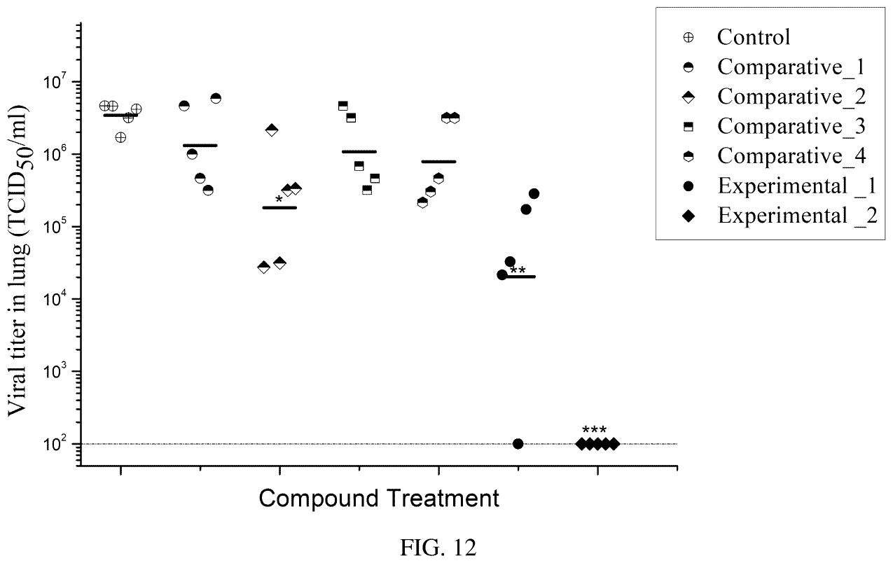

Results: referring to FIG. 12, TCID₅₀ of each of Experimental_1 and Experimental_2 and 3 was significantly lower than those of each of Comparative_1, Comparative_2, Comparative_3 and Comparative_4, revealing that the combination of isotretinoin and oligosaccharide with/without metal ions has higher in vivo efficacy against SARS-COV-2 compared to only isotretinoin, only metal ions, or only oligosaccharides.

### CONCLUSION

In view of the results of Examples 1 to 5, it is verified that the claimed novel retinoic acid compound or a pharmaceutically acceptable salt thereof, or the claimed pharmaceutical composition including the compound (i.e., the first pharmaceutical composition), or the claimed pharmaceutical composition including a retinoic acid and a carbohydrate with/without metal ions (i.e., the second pharmaceutical composition) can provide an improving and/or synergistic effect on inhibiting infection and replication of SARS-COV-2 and treating a disease associated with SARS-COV-2 infection or cancers. Consequently, the compound including the retinoic acid conjugated with the carbohydrate, the pharmaceutically acceptable salt thereof, and the first and second pharmaceutical compositions of the present invention indeed can be served as drug-repurposing agents.

The present invention has been described broadly and generically herein. Each of the narrower species and subgeneric groupings falling within the generic disclosure also form part of the present invention. In addition, where features or aspects of the present invention are described in terms of Markush groups, those skilled in the art will recognize that the present invention is also thereby described in terms of any individual member or subgroup of members of the Markush group.

What is claimed is:

1. A compound or a pharmaceutically acceptable salt thereof, wherein the compound comprises a retinoic acid conjugated with a carbohydrate.

2. The compound or the pharmaceutically acceptable salt thereof according to claim 1,
wherein the compound is represented by formula (I): 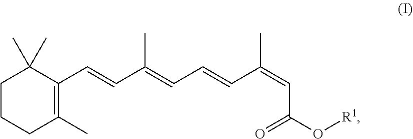
wherein R¹ is a substituted or unsubstituted functional group of the carbohydrate.

3. The compound or the pharmaceutically acceptable salt thereof according to claim 1, wherein the carbohydrate is selected from a group consisting of a monosaccharide, a disaccharide, an oligosaccharide, and a polysaccharide.

4. The compound or the pharmaceutically acceptable salt thereof according to claim 3, wherein the oligosaccharide is monooligosaccharide or heterooligosaccharide; and the polysaccharide is monopolysaccharide or heteropolysaccharide.

5. The compound or the pharmaceutically acceptable salt thereof according to claim 3, wherein the carbohydrate comprises glucose, fructose, galactose, mannose, sucrose, lactose, maltose, β-1,3/1,6-glucan oligosaccharides, raffinose, stachyose, verbascose, fructooligosaccharides, starch, glycogen, cellulose, or any combination thereof.

6. A pharmaceutical composition comprising the compound or the pharmaceutically acceptable salt thereof according to claim 1 and a pharmaceutically acceptable carrier.

7. The pharmaceutical composition according to claim 6, wherein the pharmaceutically acceptable carrier comprises a liposome, and the compound or the pharmaceutically acceptable salt thereof is encapsulated by the liposome.

8. The pharmaceutical composition according to claim 6, further comprising a metal ion.

9. The pharmaceutical composition according to claim 8, wherein the metal ion comprises a monovalent ion, a divalent ion, or a combination thereof.

10. The pharmaceutical composition according to claim 9, wherein the monovalent ion comprises K⁺, Na⁺, or a combination thereof; and the divalent ion comprises Zn²⁺, Mg²⁺, Cu²⁺, Mn²⁺, Ca²⁺, Fe²⁺, or any combination thereof.

11. The pharmaceutical composition according to claim 8, wherein the pharmaceutically acceptable carrier comprises a liposome, and the metal ion is encapsulated by the liposome.

12. The pharmaceutical composition according to claim 11, wherein the compound or the pharmaceutically acceptable salt thereof and the metal ion are individually or simultaneously encapsulated by the liposome.

13. Use of the compound or the pharmaceutically acceptable salt thereof according to claim 1 in the manufacture of a medicament for inhibiting infection or replication of a virus.

14. The use according to claim 13, wherein the virus is an RNA virus.

15. The use according to claim 14, wherein the RNA virus comprises coronavirus, human immunodeficiency virus (HIV), hepatitis C virus (HCV), influenza virus, or any combination thereof.

16. The use according to claim 15, wherein the coronavirus comprises severe acute respiratory syndrome coronavirus (SARS-COV), severe acute respiratory syndrome coronavirus 2 (SARS-COV-2), Middle East respiratory syndrome coronavirus (MERS-COV), human coronavirus 229E (HcoV-229E), human coronavirus OC43 (HCoV-OC43), human coronavirus NL63 (HCoV-NL63), human coronavirus HKU (HCoV-HKU1), or any combination thereof.

17. Use of the compound or the pharmaceutically acceptable salt thereof according to claim 1 in the manufacture of a medicament for treating a cancer.

18. The use according to claim 17, wherein the cancer comprises leukemias, lymphomas, carcinomas, or sarcomas.
